(19) United States
(12) Patent Application Publication
(10) Pub. No.: US 2008/0220083 A1
(43) Pub. Date: Sep. 11, 2008

(54) Pharmaceutical Agent Comprising Blood Components <10 Kda And Their Use For Prophylaxis And Treatment Of Defects Of The Immune System

(75) Inventor: Zoser B. Salama, Berlin (DE)

Correspondence Address: JOYCE VON NATZMER;PEQUIGNOT + MYERS LLC, 200 Madison Avenue, Suite 1901, New York, NY, 10016, US

(73) Assignee: Salama

(21) Appl. No.: 11/575,931

(22) PCT Filed: Sep. 26, 2005
(86) PCT No.: PCT/DE05/01729

§ 371 Date: Nov. 8, 2007

(30) Foreign Application Priority Data
Sep. 24, 2004 (EP) 04090376.7

(51) Int. Cl. A61K 35/14; A61P 37/00

(52) U.S. Cl. 424/529

(57) ABSTRACT

The invention relates to a composition of proteins, peptides and/or peptide components, a pharmaceutical agent comprising said composition, a method for the production of said composition and the use thereof in the prophylaxis or therapy of persons, animals and/or patients with pathogenic modifications and/or cellular immunodeficiencies, especially cancer, septicemia or allergic reactions, in connection with a cytostatic agent therapy, chemotherapy and/or radiotherapy.

The invention relates to a composition of peptide and/or protein components of the cellular constituents of blood, which components have a molecular weight of less than 10,000 Da, a pharmaceutical agent comprising said composition, the production of said composition, the use thereof in the treatment of cellular immunodeficiency, especially cancer, septicemia, allergic reactions, and the prophylactic use of the pharmaceutical agents in the treatment of a cancer patient, using e.g. cytostatic agents or high-energy radiation.

The environment of humans or animals contains a large number of infectious microbes such as viruses, bacteria, fungi, and parasites. Furthermore, disorders or modifications of the metabolism, especially of cell division, may impose the necessity on individuals of—apart from handling environmental influences—dealing with pathological processes in themselves, e.g. in case of autoimmunity, cancerous diseases, or benign polyp formation. These processes, i.e., handling exterior and interior influences, are generally subject to interaction as is the case e.g. in inflammatory reactions reflecting a reaction to pathogens possibly invading individuals from the environment, causing proliferation of cells.

The above-mentioned processes may cause pathological damage and, in the event of uncontrolled interaction with the defense system of the organism, are capable of killing the individual, i.e., the host. Normally, the struggle with pathogens spans a limited interval, yet may cause permanent damage in the organism even within such a limited interval. Such limitation of the time of these reactions is due to the immune system, in particular, being the defense system of the individual.

Immune responses of an individual to environmental influences or to pathogenic changes within the individual can be classified into two major groups: humoral and cellular immune responses, though unambiguous assignment of a particular process of disease or convalescence to either of the two immune responses frequently cannot be made because these processes interact, being dependent on one another. The term “cell-mediated immunity” originally was coined for local reactions to organisms, normally intracellular pathogens, which are predominantly caused by lymphocytes and phagocytes, and to a lesser extent by antibodies being part of the humoral immunity. Meanwhile, however, the term cell-mediated immunity is used for any immune response in which antibodies do not play a central role. Cell-mediated and antibody-mediated reactions cannot be regarded separately because cells are also involved in the formation of anti-bodies, for example, the antibodies assuming the function of a mediating element in numerous cell-mediated reactions.

Indeed, it is unlikely that cell-mediated reactions in the meaning of the invention could be initiated if antibodies capable of affecting cellular reactions in various ways are absent or present in only suboptimum amounts.

More specifically, the cellular immune response is associated with macrophages, B cells, T cells, lymphocytes, NK cells, monocytes and many others.

The above-mentioned cells are responsible for the liberation of cytokines such as TNF, M-CSF or GM-CSF. The combined action of cells, cytokines, as well as environmental influences and reactions of the individual himself, e.g. humoral immunity, results in:
microbicidal and tumoricidal activity,
inflammatory reactions and fever,
activation of lymphocytes, and
tissue reorganization and tissue lesion.

This may result in the destruction of microorganisms, multicellular parasites, but also destruction of tumor cells, and in febrile reactions.

In view of the diverse functions of this part of the immune defense, there were numerous attempts of increasing the activity of this system. Improving the efficiency of this part of the immune defense in a prophylactic fashion appeared particularly advantageous e.g. in those cases where a patient had a serious accident, or a major surgery was intended, or in those cases where a patient had been treated with cytostatic agents or radiotherapy. The assumption was that improving the cellular immune response would prevent new infections or additional metastases or have a favorable influence on septic and inflammatory processes.

Particularly in the treatment of autoimmune diseases such as psoriasis, atopic eczemas, rheumatoid arthritis or juvenile diabetes, good therapeutic options were thought to be available, provided the cellular immune response in a patient would be improved.

Especially with cancer, the increasing knowledge as to the role of the cellular immune system has furnished new therapeutic approaches. Using so-called “cancer vaccinations”, attempts have been made to direct the endogenous cellular defense system against tumor cells in a well-aimed fashion. The aim of previous cellular immune therapies in cases of cancer has been to eliminate the obstruction of the immune system by the tumor and activate cytotoxic T cells against the tumor. However, these processes are still associated with a risk of autoimmune reactions.

Well-known methods comprise initial isolation of immune cells from the blood or bone marrow, growth in a test tube outside the body, and return into the patient. This can be done using cells from the own body (autologous cells) or cells from a foreign donor (allogenic cells). The greater the difference between cells from donor and recipient, the higher the probability that the transplanted cells are capable of recognizing tumor cells.

Another way of increasing particularly the cellular immune response is administration of dendritic cells. Dendritic cells assume a key function in activating an immune response. They present exceptional features distinguishing tumor cells from other cells to the immune system in such a way that marked reaction can take place which involves more than just single cells.

The dendritic cells are taken from a particular patient, combined with tumor cells or parts thereof in a well-directed fashion, and subsequently returned into the patient. In the body, the dendritic cells loaded in this way are intended to present tumor cell fragments, thereby triggering an immune reaction against the tumor.

Transplantation of stem cells from bone marrow or blood is another option of cellular immune therapy. Stem cell transplantation originally has been introduced to renew leukemia patients' bone marrow destroyed by high-dosage chemotherapeutical treatment or irradiation. However, it has been found that cells transplanted from a foreign donor also have a direct effect against cancer cells precisely because of the fact that they virtually never conform in all of their tissue characteristics with those of the recipient. Now, the patient's so-called new cellular immune system formed by the donor cells therefore recognizes remaining leukemia cells as foreign cells and combats them.

However, the activation of a cellular immune response is a highly regulated process and requires a high level of biochemical energy and, in addition, is associated with the risk of an autoimmune reaction. Such clinical interventions therefore involve a risk of disadvantages and side-effects to the patient.

In addition, various agents have been disclosed in the art, which are obtained from blood products and can be used for the therapeutic activation of the immune system. WO 89/06538 A discloses compositions of whole blood, which are subjected to papain hydrolysis and alcohol denaturation. With the compositions according to WO 89/06538, wound healing is possible under certain conditions only, because the compounds have quite a number of side effects.

In the description of the prior art, U.S. Pat. No. 4,384,991 discloses about 25 different cell extracts, essentially all of which are obtained from white blood cells. That is to say, a person skilled in the art can infer from this patent the existence of a variety of compositions obtained from such cells. Above all, the compositions according to U.S. Pat. No. 4,384,991 differ from each other in their degree of purity resulting from the respective production process or the starting material thereof. In a particularly advantageous manner, horse or calf leukocytes are used as starting material. Cell extracts from the starting materials from horses and calves are suitable in obtaining therapeutic products. However, the essence of the teaching according to U.S. Pat. No. 4,384,991 is the isolation of a pure single peptide. This peptide is characterized as a high-purity molecule via so-called fingerprint features. High-purity isolation is achieved by means of cleaning steps such as ion exchange chromatography or paper electrophoresis. However, the pure peptide from horse or calf leukocytes fails to show any particular effects in the connection with use in therapy.

While the raw extracts containing the disclosed peptide also have particular effects, the required amount of this fraction is so high that a person skilled in the art cannot use them in practice. Furthermore, the absence of toxic effects has been described for the purified peptide only, so that it must be assumed that the raw extracts from which the peptide is obtained do not have such advantageous properties.

The raw fraction according to U.S. Pat. No. 4,384,991 is washed with acetone and subsequently freeze-dried. The acetone treatment (as does hydrolysis and alcohol treatment in WO 89/06538) results in structural changes of the protein components of the raw fraction. In particular, the isolation of the desired peptide is achieved by accumulation on granulocytes, i.e., separation of lymphocytes and monocytes is effected. The granulocyte extract from horse or calf blood cannot be used in a purposeful fashion in practice because it seems to have specific effects at high concentrations only and, in addition, has toxic effects.

EP 0 140 134 discloses an extract from organs of mammals or from cell cultures. In addition, extracts of cell cultures obtained from fresh calf blood or calf blood serum are disclosed therein. Essentially, the disclosed compositions are calf blood serum, fresh calf blood or defibrinated calf blood lacking components having a size of more than 10,000 Da. Such extracts give rise to undesirable defense reactions in a target organism.

The object of the present invention is therefore to provide a technical teaching which would not involve the above-mentioned drawbacks of the prior art, permitting easy, safe and efficient treatment of diseases particularly resulting from deficiencies of the cellular immune response, such as septicemia, tumor diseases, eczemas, psoriasis, neurodermitis and autoimmune diseases. Another object of the present invention is to provide a technical teaching, particularly a pharmaceutical agent which can be administered to a patient in a prophylactic fashion in cases of severe accidents, but also in a treatment using chemotherapeutical agents and/or radiation in order to optimize the patient's general condition preferably via improvement of the cellular immune response. Still another object of the invention is to provide an agent for prophylactic use and/or for the treatment of fatigue syndrome as a result of shock and/or physical, emotional, nervous, pathological or radioactive effects.

The technical object of the invention is accomplished by means of a method according to claim 1, particularly for the production of a composition comprising complete and/or fractions of ubiquitin-specific protease 32, positive cofactor 2 glutamine/Q-rich associated protein, cadherin, transcription factor GATA-2, putative chromatin structure regulator, CUE domain-containing 1, SUPT6H protein, interleukin-18 receptor 1 precursor, interleukin-1 receptor-like protein and mucin 4 and/or tenascin M, transient receptor potential cation channel, ectonucleotide pyrophosphatase/-phosphodiesterase, SWI/SNF-related matrix-associated actin-dependent regulator of chromatin, SWI/SNF chromatin-modulating complex subunit OSA1 B120, OSA1 nucleoprotein, AT-Pase, H⁺/K⁺ change polypeptide, zinc-finger protein 174, Ig heavy chain V region, ADAMTS-3 precursor/desintegrin-like and metalloprotease, coagulation factor II (thrombin) receptor-like 3, diacylglycerol kinase-theta, p250R, SWI/SNF chromatin-remodulating complex subunit OSA2, metallo-phospoesterase, AMP deaminase, α-mannosidase, cadherin-9, Nik-related kinase, α-1B-adrenergic receptor, BRCA1-associated protein, CD99 antigen-like 2 isoform E3-E4, SWHCF-comprising peptide and/or FAT-like cadherin-FATJ, ATP binding cassette, nucleoporin 153 kDa, ELK3 protein, protein ELK-3 ETS domain, ATPase, copper-transporting protein kinase, protein-tyrosine phosphatase, wingless type MMTV integration site family, MYC binding protein 2, cullin 7, dissolved carrier family 5 (sodium iodide symporter) member 5, glutamate-rich WD repeat containing 1, MAP kinase-interacting serine/threonine kinase 1, ATP binding cassette, NOV plexin A1 protein and/or E3 ubiquitin ligase SMURF2, acyl-CoA synthetase, estrogen sulfotransferase, 2,4-dienoyl CoA reductase 1 precursor, 4-enoyl CoA reductase, claudin 10 isoform b, DMBT1, extracellular linker domain containing 1, lymphatic vessel endothelial cell-specific hyaluron receptor LYVE-1, Rho-GTPase-activating protein 8 isoform 2, desintegrin-like and metalloprotease (reprolysine type) with thrombospondin type 1 motif, Ig heavy chain V region, AS12 protein, mitochondrial ribosomal protein S9, 28S ribosomal protein S9, protein kinase substrate MK2S4, NP220, putative G protein-coupled receptor, dynein, axonemal heavy polypeptide 5, N-acylphosphatidyl ethanolamine-hydrolyzing phospholipase D, leukotriene B4 receptor, G protein-coupled receptor 16, proprotein convertase subtilisin/kexin type 1 inhibitor CMKRL1, dual-specific tyrosine phosphorylation-regulated kinase 3, regulatory erythroid kinase (long form), DYRK3 protein, Ig lambda chain V-VII region (Mot)—human, glutathione reductase, mitochondrial precursor, collagen alpha 1 (XVI) chain precursors, 11-β-hydroxysteroid dehydrogenase 1, insulin receptor substrate 2, Vault poly(ADP-ribose) polymerase (VPARP), calcium/calmodulin-dependent 3′,5′-cyclic nucleotides, zinc-finger protein 161, H2.0-like homeobox-1, H2.0 (drosophila)-like homeobox-1 and/or dedicator of cytokinesis 6.

Most surprisingly, the method according to the invention allows to obtain a composition from the above-mentioned cellular blood components, especially from leukocytes, which composition can be used in the prophylaxis and therapy of pathogenic modifications of cellular immunity and does not have the disadvantages of the prior art. The individual process steps result in denaturation products and cleaved products having advantageous properties. The method and composition according to the invention act to solve a hitherto unresolved urgent issue. To date, the art has seen futile efforts of solving the problem of pathogenic modification in cellular immunity. The teaching of the invention suggests a simple solution to this problem. Up to now, the development in scientific technology has proceeded in a different direction (see explanations relating to the state of the art). The teaching according to the present application therefore represents an achievement in short-term development, eliminating erroneous ideas on the solution to the above-mentioned problem in the art. In particular, the technical progress achieved by means of the teaching according to the invention becomes apparent in an improvement and enhanced performance of the method and the products obtained therefrom, lower cost, saving of time, material, process steps, expenses and starting materials difficult to obtain, in an improved reliability, elimination of errors, in improved quality, in an enhanced prophylactic and therapeutic potential, but also in the provision of an additional agent and in furnishing an additional method of obtaining agents which could be used in pathogenic modifications of cellular immunity, and ultimately, the range of available drugs is enriched by the teaching according to the present application.

In a preferred embodiment the composition comprises, among other things, complete and/or fractions of interleukin-18 receptor 1 precursor, interleukin-1 receptor-like protein and mucin 4, transient receptor potential cation channel, ectonucleotide pyrophosphatase/phosphodiesterase, SWI/SNF-related matrix-associated actin-dependent regulator of chromatin, SWI/SNF chromatin-modulating complex subunit OSA1 B120, OSA1 nucleoprotein, MYC binding protein 2, cullin 7, dissolved carrier family 5 (sodium iodide symporter) member 5, glutamate-rich WD repeat containing 1, MAP kinase-interacting serine/threonine kinase 1, ATP binding cassette, DMBT1, extracellular linker domain containing 1, lymphatic vessel endothelial cell-specific hyaluron receptor LYVE-1, Rho-GTPase-activating protein 8 isoform 2, desintegrin-like and metalloprotease (reprolysine type) with thrombospondin type 1 motif, AS12 protein, mitochondrial ribosomal protein S9, 28S ribosomal protein S9, protein kinase substrate MK2S4, NP220, putative G protein-coupled receptor, dynein, axonemal, heavy polypeptide 5, N-acylphosphatidyl ethanolamine-hydrolyzing phospholipase D, leukotriene B4 receptor, G protein-coupled receptor 16, proprotein convertase subtilisin/kexin type 1 inhibitor CMKRL1, dual-specific tyrosine phosphorylation-regulated kinase 3, regulatory erythroid kinase (long form), DYRK3 protein, Ig lambda chain V-VII region (Mot)—human.

Surprisingly, the composition or the preparation according to the present application can be used for prophylactic and therapeutic purposes and in supporting biological activities. For therapeutic purposes, the composition can be used as a pharmaceutical agent, particularly in the form of a combination of composition and pharmaceutically acceptable carrier, to treat chronic infections, septic infections, atopic eczemas, neurodermitis, psoriasis and others of the above-mentioned immune diseases. For example, a prophylactic indication of the composition is administration of the composition to a patient being treated with a radiotherapy or chemotherapy using cytostatic agents in order to prevent or alleviate the immune suppression initiated by such types of therapy. Prophylactic administration of the composition according to the invention also makes sense during the pre-surgical phase of patients, e.g. in those cases where blood transfusion gives rise to cellular intolerance which disadvantageously changes the immune status of a patient. Obviously, the immune status of a patient can also be adversely changed as a result of accidents, major or minor injuries, or following traumas, so that direct therapy is not possible immediately. In this event, the composition of the invention can also be used in a prophylactic fashion in order to stabilize the condition of the patient in such a way that injuries or pathogenic changes can be put to causal therapy.

More specifically, the composition can be used in a supporting and therapy-associated fashion in those cases where proliferation and differentiation of different cell types at different stages of maturing is to be optimized, liberation of CD₄ or CD₈ and IF-gamma increased, or the activity of T lymphocytes improved.

Another possible use is activation of thymocyte populations (Th 1) or liberation of cytokines and interleukins. Furthermore, it is possible to activate the transport of calcium ions through the cell membrane or to improve the oxidative metabolism in cells of important metabolic organs such as the liver or kidneys. Moreover, regulatory suppression mechanisms of immunologic cascades can be activated.

Of course, the composition according to the invention may also comprise conventional auxiliary agents, preferably carriers, adjuvants and/or vehicles. For example, said carriers can be fillers, extenders, binders, humectants, disintegrants, dissolution retarders, absorption enhancers, wetting agents, adsorbents, and/or lubricants. In this event, the composition more specifically is referred to as drug or pharmaceutical agent.

In another preferred embodiment of the invention the inventive agent is prepared as a gel, powder, tablet, sustained-release tablet, premix, emulsion, infusion formulation, drops, concentrate, granulate, syrup, pellet, bolus, capsule, aerosol, spray and/or inhalant and/or used in this form. The tablets, coated tablets, capsules, pills and granulates can be provided with conventional coatings and envelopes optionally including opacification agents, and can be composed such that release of the active substance(s) takes place only or preferably in a particular part of the intestinal tract, optionally in a delayed fashion, to which end polymer substances and waxes can be used as embedding materials.

For example, the drugs of the present invention can be used in oral administration in any orally tolerable dosage form, including capsules, tablets and aqueous suspensions and solutions, without being restricted thereto. In case of tablets for oral application, carriers frequently used include lactose and corn starch. Typically, lubricants such as magnesium stearate can be added. For oral administration in the form of capsules, useful diluents such as lactose and dried corn starch are employed. In oral administration of aqueous suspensions the active substance is combined with emulsifiers and suspending agents. Also, particular sweeteners and/or flavors and/or coloring agents can be added, if desired.

The active substance(s) can also be present in micro-encapsulated form, optionally with one or more of the above-specified carriers.

In addition to the active substance(s), suppositories may include conventional water-soluble or water-insoluble carriers such as polyethylene glycols, fats, e.g. cocoa fat and higher esters (for example, C₁₄ alcohols with C₁₆ fatty acids) or mixtures of these substances.

In addition to the active substance(s), ointments, pastes, creams and gels may include conventional carriers such as animal and vegetable fats, waxes, paraffins, starch, tragacanth, cellulose derivatives, polyethylene glycols, silicones, bentonites, silicic acid, talc and zinc oxide or mixtures of these substances.

In addition to the active substance(s), powders and sprays may include conventional carriers such as lactose, talc, silicic acid, aluminum hydroxide, calcium silicate and polyamide powder or mixtures of these substances. In addition, sprays may include conventional propellants such as chlorofluorohydrocarbons.

In addition to the active substance(s), i.e., the composition according to the invention, solutions and emulsions may include conventional carriers such as solvents, solubilizers, and emulsifiers such as water, ethyl alcohol, isopropyl alcohol, ethyl carbonate, ethyl acetate, benzyl alcohol, benzyl benzoate, propylene glycol, 1,3-butylene glycol, dimethylformamide, oils, especially cotton seed oil, peanut oil, corn oil, olive oil, castor oil and sesame oil, glycerol, glycerol formal, tetrahydrofurfuryl alcohol, polyethylene glycols, and fatty esters of sorbitan, or mixtures of these substances. For parenteral application, the solutions and emulsions may also be present in a sterile and blood-isotonic form.

In addition to the active substance(s), suspensions may include conventional carriers such as liquid diluents, e.g. water, ethyl alcohol, propylene glycol, suspending agents, e.g. ethoxylated isostearyl alcohols, polyoxyethylenesorbitol and sorbitan esters, microcrystalline cellulose, aluminum metahydroxide, bentonite, agar, and tragacanth, or mixtures of these substances.

The drugs can be present in the form of a lyophilized sterile injectable formulation, e.g. as a sterile injectable aqueous or oily suspension. Such a suspension can also be formulated by means of methods known in the art, using suitable dispersing or wetting agents (such as Tween 80) and suspending agents. The sterile injectable formulation can also be a sterile injectable solution or suspension in a non-toxic, parenterally tolerable diluent or solvent, e.g. a solution in 1,3-butanediol. Tolerable vehicles and solvents that can be used include mannitol, water, Ringer's solution, and isotonic sodium chloride solution. Furthermore, sterile, non-volatile oils are conventionally used as solvents or suspending medium. Any mild non-volatile oil, including synthetic mono- or diglycerides, can be used for this purpose. Fatty acids such as oleic acid and glyceride derivatives thereof can be used in the production of injection agents, e.g. natural pharmaceutically tolerable oils such as olive oil or castor oil, especially in their polyoxyethylated forms. Such oil solutions or suspensions may also include a long-chain alcohol or a similar alcohol as diluent or dispersant.

The above-mentioned formulation forms may also include colorants, preservatives, as well as odor- and taste-improving additives, e.g. peppermint oil and eucalyptus oil, and sweeteners, e.g. saccharine. Preferably, the composition according to the invention should be present in the above-mentioned pharmaceutical formulations at a concentration of about 0.01 to 99.9, more preferably about 0.05 to 99 wt.-% of the overall mixture.

In addition to the compositions, the above-mentioned pharmaceutical formulations may include further pharmaceutical active substances, but also, in addition to said further pharmaceutical active substances, salts, buffers, vitamins, sugar derivatives, especially saccharides, enzymes, vegetable extracts and others. Buffers and sugar derivatives advantageously reduce the pain during subcutaneous application, and enzymes such as hyaluronidase increase the effectiveness. The production of the pharmaceutical formulations specified above proceeds in a usual manner according to well-known methods, e.g. by mixing the active substance(s) with the carrier(s).

The above-mentioned formulations can be applied in humans and animals on an oral, rectal, parenteral (intravenous, intramuscular, subcutaneous), intracisternal, intravaginal, intraperitoneal route or locally (powders, ointment, drops) and used in the therapy of the diseases specified below. For oral therapy, injection solutions, solutions and suspensions, gels, infusion formulations, emulsions, ointments or drops are possible as suitable formulations. For local therapy, ophthalmic and dermatological formulations, silver and other salts, ear drops, eye ointments, powders or solutions can be used. With animals, ingestion can be effected via feed or drinking water in suitable formulations. Furthermore, the drugs can be incorporated in other carrier materials such as plastics—plastic chains for local therapy—collagen or bone cement.

In another preferred embodiment of the invention the composition is incorporated in a pharmaceutical formulation at a concentration of 0.1 to 99.5, preferably 0.5 to 95, and more preferably 20 to 80 wt.-%. That is, the composition is present in the above pharmaceutical formulations, e.g. tablets, pills, granulates and others, at a concentration of preferably 0.1 to 99.5 wt.-% of the overall mixture. Those skilled in the art will be aware of the fact that the amount of active substance, i.e., the amount of inventive composition combined with the carrier materials to produce a single dosage form, will vary depending on the patient to be treated and on the particular type of administration. Once the condition of the patient has improved, the proportion of active compound in the formulation can be modified so as to obtain a maintenance dose that will bring the disease to a halt. Depending on the symptoms, the dose or frequency of administration or both can subsequently be reduced to a level where the improved condition is retained. Once the symptoms have been alleviated to the desired level, the treatment should be stopped. However, patients may require an intermittent treatment on a long-term basis if any symptoms of the disease should recur. Accordingly, the proportion of the composition, i.e., its concentration, in the overall mixture of the pharmaceutical formulation, as well as the composition or combination thereof, is variable and can be modified and adapted by a person of specialized knowledge in the art.

Those skilled in the art will be aware of the fact that the composition of the invention can be contacted with an organism, preferably a human or an animal, on various routes. In particular, an artisan will also be familiar with the fact that the pharmaceutical agents can be applied at varying dosages. Application should be effected in such a way that a disease is combated as effectively as possible, or the onset of a disease is prevented by a prophylactic administration. Concentration and type of application can be determined by a person skilled in the art using routine tests. Preferred applications of the compounds of the invention are oral application in the form of powders, tablets, juice, drops, capsules or the like, rectal application in the form of suppositories, solutions and the like, parenteral application in the form of injections, infusions and solutions, and local application in the form of ointments, pads, dressings, lavages and the like. Contacting with the composition according to the invention is preferably effected in a prophylactic or therapeutic fashion.

For example, the suitability of the selected form of application, of the dose, application regimen, selection of adjuvant and the like can be determined by taking serum aliquots from the patient, i.e., human or animal, and testing for the presence of indicators of a disease in the course of the treatment procedure. Alternatively or concomitantly, the condition of the kidneys, liver and the like, but also, the amount of T cells or other cells of the immune system, can be determined in a conventional manner so as to obtain a general survey on the immunologic constitution of the patient and, in particular, the constitution of organs important to the metabolism. Additionally, the clinical condition of the patient can be observed for the desired effects. Where insufficient therapeutic effectiveness results, the patient can be subjected to further treatment using the agents of the invention, optionally modified with other well-known medicaments expected to bring about an improvement of the overall constitution. Obviously, it is also possible to modify the carriers or vehicles of the pharmaceutical agent or to vary the route of administration.

In addition to oral ingestion, intramuscular or subcutaneous injections, or injections into the blood vessels can be envisaged as another preferred route of therapeutic administration of the composition according to the invention. At the same time, influx via catheters or surgical tubes can also be used, e.g. via catheters directly leading to particular organs such as kidneys, liver, spleen, intestine, lungs, etc.

In a preferred embodiment, the composition of the invention can be employed in a total amount of preferably 0.05 to 500 mg/kg body weight per 24 hours, more preferably 5 to 100 mg/kg body weight. Advantageously, this is a therapeutic quantity which is used to prevent or improve the symptoms of a disorder or responsive, pathological physiological condition.

Obviously, the dose will depend on the age, health and weight of the recipient, degree of the disease, type of required simultaneous treatment, frequency of the treatment and type of the desired effects, and side-effects. The daily dose of 0.05 to 500 mg/kg body weight can be applied as a single dose or multiple doses in order to furnish the desired results. In particular, pharmaceutical agents are typically used in about 1 to 10 administrations per day, or alternatively or additionally as a continuous infusion. Such administrations can be applied as a chronic or acute therapy. Of course, the amounts of active substance that are combined with the carrier materials to produce a single dosage form may vary depending on the host to be treated and on the particular type of administration. In a preferred fashion, the daily dose is distributed over 2 to 5 applications, with 1 to 2 tablets including an active substance content of 0.05 to 500 mg/kg body weight being administered in each application. Of course, it is also possible to select a higher content of active substance, e.g. up to a concentration of 5000 mg/kg. The tablets can also be sustained-release tablets, in which case the number of applications per day is reduced to 1 to 3. The active substance content of sustained-release tablets can be from 3 to 3000 mg. If the active substance—as set forth above—is administered by injection, the host is preferably contacted 1 to 10 times per day with the composition of the invention or by using continuous infusion, in which case quantities of from 1 to 4000 mg per day are preferred. The preferred total amounts per day were found advantageous both in human and veterinary medicine. It may become necessary to deviate from the above-mentioned dosages, and this depends on the nature and body weight of the host to be treated, the type and severity of the disease, the type of formulation and application of the drug, and on the time period or interval during which the administration takes place. Thus, it may be preferred in some cases to contact the organism with less than the amounts mentioned above, while in other cases the amount of active substance specified above has to be surpassed. A person of specialized knowledge in the art can determine the optimum dosages required in each case and the type of application of the active substances.

In another particularly preferred embodiment of the invention the pharmaceutical agent is used in a single administration of from 1 to 100, especially from 2 to 50 mg/kg body weight. In the same way as the total amount per day (see above), the amount of a single dose per application can be varied by a person of specialized knowledge in the art. Similarly, the compounds used according to the invention can be employed in veterinary medicine with the above-mentioned single concentrations and formulations together with the feed or feed formulations or drinking water. A single dose preferably includes that amount of active substance which is administered in one application and which normally correspond to one whole, one half daily dose or one third or one quarter of a daily dose. Accordingly, the dosage units may preferably include 1, 2, 3 or 4 or more single doses or 0.5, 0.3 or 0.25 single doses. In a preferred fashion, the daily dose of the compounds according to the invention is distributed over 2 to 10 applications, preferably 2 to 7, and more preferably 3 to 5 applications. Of course, continuous infusion of the agents according to the invention is also possible.

In a particularly preferred embodiment of the invention, 1 to 2 tablets are administered in each oral application of the compounds of the invention. The tablets according to the invention can be provided with coatings and envelopes well-known to those skilled in the art or can be composed in a way so as to release the active substance(s) only in preferred, particular regions of the host.

In another embodiment of the invention the single components of the composition are preferably associated with each other or, coupled to a carrier, enclosed in liposomes, and such enclosure in liposomes does not necessarily imply—in the meaning of the invention—that the composition is present inside the liposomes. Enclosure in the meaning of the invention may also imply that the composition is associated with the membrane of the liposomes, e.g. in such a way that the composition is anchored on the exterior of the membrane. Such a representation of the inventive composition in or on liposomes is advantageous in those cases where a person skilled in the art selects the liposomes such that the latter have an immunostimulant effect. Various ways of modifying the immunostimulant effect of liposomes are known to those skilled in the art from DE 198 51 282. The lipids can be ordinary lipids, such as esters and amides, or complex lipids, e.g. glycolipids such as cerebrosides or gangliosides, sphingolipids or phospholipids.

The invention also relates to a method for the production of a composition which can be used for the above-mentioned prophylactic and therapeutic indications and for therapy-associated improvements of the biological efficiency, especially of the cellular immune system.

The method according to the invention comprises collecting and homogenizing blood, plasma or serum components causing no problems with immunologic tolerance or to the least possible extent. For example, homogenizing can be effected mechanically and/or by means of freezing/thawing cycles or other homogenizing methods known to those skilled in the art. Furthermore, initial freezing and subsequent cutting, e.g. with a microtome, of the starting components used to produce the composition or of the composition itself can be advantageous.

Homogenization in the meaning of the invention encompasses all procedures capable of inducing or supporting cell lysis. Homogenizing enables intimate mixing of per se immiscible or sparsely miscible components of a system across the entire volume, so that the material obtained, largely independent of the number of components, essentially exhibits only a few phases, and particularly a single phase. Homogenization results in a reduction in particle size of the dispersed phase, deagglomeration of particle aggregates, and provides dispersions with increased sedimentation stability. Such homogenization can be effected using dynamic apparatus, as well as static apparatus, i.e., in mixers without moving parts. Preferred starting materials are blood cells, preferably white blood cells. For example, the blood cells can be prepared in the form of a buffy coat can rich in thrombocytes and erythrocytes. Standard procedures of producing such cans are well-known to those skilled in the art. Obviously, the homogenized sample can also be obtained in the form of a leukocyte concentrate.

Furthermore, initial preparation of the starting material, e.g. red and/or white blood cells, in the form of a buffy coat can, followed by homogenization of the biologically active components, e.g. by means of a freezing/thawing cycle, may also be advantageous. That is, the precise sequence of each single step of the procedure is exchangeable in the meaning of the invention. Using dialysis, centrifugation and/or filtration, substantially all components larger than 10,000 Da are removed from the product obtained by the freezing/thawing cycle or other homogenization procedures. Removal of any components with a size of more than 3,000 Da can be particularly preferred. It is particularly those compositions which have the above-mentioned advantageous properties.

Concentrating the product obtained by dialysis, centrifugation or filtration can be advantageous. In an advantageous embodiment of the invention, cellular blood components such as leukocytes are first homogenized and subsequently dialyzed, followed by lyophilization. Advantageously, a solution can be prepared thereafter, which initially is prefiltrated, then ultrafiltrated and subsequently subjected to sterile filtration.

The filtrate obtained can be pasteurized in a water bath, for example. Following this process, the pasteurized material can be sterilized and re-lyophilized. Of course, other sterilization methods can also be used, e.g. treatment with high-energy radiation such as UV radiation or X-rays. Each of the above-mentioned single steps can be accompanied by quality controls. For example, this can be done by taking aliquots from the sample and testing the aliquots for the presence of microorganisms, viruses or other undesirable components.

The blood cell concentrate, especially the leukocyte concentrate, is preferably prepared using freezing/thawing cycles or ultrasonic treatment of the cells or a combination of both procedures.

Especially by means of the freeze-thaw cycle it is possible to obtain stable and reproducible fragments from the above-mentioned proteins and peptides of the cellular components of blood. The composition according to the invention represents the lyophilizable, sterilizable, filtratable and dialyzable homogenate from blood cells, especially white blood cells, having undergone several freeze-thaw cycles and sterilization at temperatures of about 100° C. The freeze-thaw cycle can be designed in such a way that the frozen material is cut, e.g. with a microtome, to be subsequently thawed and optionally re-frozen. In a preferred fashion the freeze-thaw cycle is thus a freeze-cut-thaw cycle. The process conditions are selected such that only stable fragments of the peptides and proteins are present in the composition obtained.

Dialysis of the homogenized product is effected in such a way that low-molecular weight particles of colloids or macromolecules migrate by diffusion from the homogenized product through a semipermeable membrane and into a preferably flowing, pure solvent, thereby retaining large molecules. The rate of dialysis can be increased by raising the temperature or by applying an electric voltage as in electrodialysis, for example. Of course, dialysis can also be effected using dialysis columns, thereby removing molecules with a molecular weight of more than 10 kDa.

In particular, freeze-drying is used to concentrate the dialyzed product. Freeze-drying in the meaning of the invention is a term that describes drying of a frozen material in a high vacuum by freezing out the solvent which undergoes evaporation in the frozen state by sublimation drying. Freeze-drying in the meaning of the invention can also be effected as a dehydration, particularly by using solutions, and preferably by adding solutions such as serum, milk, carbohydrates, amino acids, enzymes, buffer solutions, salts and/or vitamins. To re-dissolve the lyophilized material for use, it is possible to dissolve in distilled water or other solvents.

Once the above solution has been prepared, determination of an ultraviolet spectrum of the material is recommended, particularly in the region of 200 nm and 400 nm. If the material is free of undesirable components or largely free of such components, pre-filtration e.g. through a Millipore membrane is advantageous. In this procedure, solid particles are separated from liquids. A porous medium is used, e.g. a Millipore membrane with a pre-filter, through which the continuous phase of the liquid is flowing, and simultaneously, the dispersed phase is retained on the surface of the porous agent or in the inside thereof.

Material with a limit value of 10 kDa can be removed using ultrafiltration. Advantageously, the material obtained can be sterilized by means of an additional filtration step, using a sterilization filter, for example. Ultrafiltration can be effected using membrane microfiltration or reversed osmosis. For ultrafiltration, porous membranes of asymmetrical structure are mostly used, which are made of various organic and inorganic materials such as polysulfone or ceramics in the form of tubes, capillaries, hollow fibers and flat membranes.

Advantageously, the sterilized material thus obtained is pasteurized using inactivation by heat. For example, pasteurization can be effected in a water bath at a temperature of 60° C. for several hours. Of course, pasteurization can be performed at any temperature below 100° C., and in particular cases above 100° C. for any desired period of time. That is, sterilization at more than 100° C. is also pasteurization in the meaning of the invention.

Advantageously, such sterilization or pasteurization at more than 100° C. results in reproducible, stable, well-usable denaturation products and cleavage products which do not have the disadvantages of prior art compositions. In particular, this applies to those cases where the cellular starting materials are human leukocytes. Most surprisingly, the above-mentioned astonishing advantages can be achieved by combining the features: (i) human leukocytes as starting materials, (ii) which are treated using the method according to the invention and/or (iii) pasteurization at more than 100° C. In a particularly advantageous manner the starting materials are not subjected to papain hydrolysis or alcoholic denaturation. It is also advantageous to do without drastic cleaning steps as encountered e.g. in ion exchange chromatography or paper electrophoresis because they result in other products barely usable in therapy. Furthermore, the absence of red blood cells is advantageous. Surprisingly, minor modifications to the method, such as initial denaturation with organic solvents, result in completely different products of the process, which cannot be used to solve the problem of the invention. It is only in some particular cases where non-human leukocytes can be used to accomplish the object of the invention. Furthermore, removal of any components with a size of more than 3,000 Da is advantageous.

The invention also relates to the use of the composition and/or pharmaceutical agent of the invention in the treatment of diseases associated with cellular immunodeficiency, e.g. a deficiency according to ICD10 Code: D.84.4. These can be septicemic diseases, inflammatory reactions and fevers, autoimmune diseases, and diseases associated with cell division disorders, such as cancer.

Inflammations in the meaning of the invention are reactions of the organism, mediated by the connective tissue and blood vessels, to an external or internally triggered inflammatory stimulus, with the purpose of eliminating or in-activating the latter and repairing the tissue lesion caused by said stimulus. A triggering effect is caused by mechanical stimuli (foreign bodies, pressure, injury) and other physical factors (ionizing radiation, UV light, heat, cold), chemical substances (alkaline solutions, acids, heavy metals, bacterial toxins, allergens, and immune complexes), and pathogens (microorganisms, worms, insects), or pathologic metabolites, derailed enzymes, malignant tumors. The process begins with a brief arteriolar constriction (as a result of adrenaline effect), with inadequate circulation and tissue alteration, followed by development of classical local inflammatory signs (cardinal symptoms, according to GALEN and CELSUS), i.e., from reddening (=rubor; vascular dilation caused by histamine), heat (=calor; as a result of local increase of metabolism), swelling (=tumor; as a result of secretion of protein-rich liquor from vessel walls changed by histamine, among other things, supported by decelerated blood circulation in the sense of a prestasis up to stasis), pain (=dolor; as a result of increased tissue tension and algogenic inflammation products, e.g. bradykinin), and functional disorders (=functio laesa). The process is accompanied by disorders in the electrolyte metabolism (transmineralization), invasion of neutrophilic granulocytes and monocytes through the vessel wall (cf., leukotaxis), with the purpose of eliminating the inflammatory stimulus and the damaged to necrotic cells (phagocytosis); furthermore, invasion of lymphocyte effector cells, giving rise to formation of specific antibodies against the inflammatory stimulus (immune reaction), and of eosinophiles (during the phase of healing or—at a very early stage—in allergic-hyperergic processes). As a result of the activation of the complement system occurring during the reaction, fragments (C3a and C5a) of this system are liberated which—like histamine and bradykinin—act as inflammation mediators, namely, in the sense of stimulating the chemotaxis of the above-mentioned blood cells; furthermore, the blood coagulation is activated. As a consequence, damage (dystrophia and coagulation necrosis) of the associated organ parenchyma occurs. Depending on the intensity and type of the inflammation, the overall organism responds with fever, stress (cf., adaptation syndrome), leukocytosis and changes in the composition of the plasma proteins (acute-phase reaction), giving rise to an accelerated erythrocyte sedimentation. Preferred inflammations in the meaning of the invention are suppurative, exudative, fibrinous, gangrenescent, granulomatous, hemorrhagic, catarrhal, necrotizing, proliferative or productive, pseudomembranous, serous, specific and/or ulcerous inflammations.

Autoimmune diseases in the meaning of the invention are diseases entirely or partially due to the formation of autoantibodies and their damaging effect on the overall organism or organ systems, i.e., due to autoaggression. A classification into organ-specific, intermediary and/or systemic autoimmune diseases can be made. Preferred organ-specific autoimmune disease are HASHIMOTO thyroiditis, primary myxedema, thyrotoxicosis (BASEDOW disease), pernicious anemia, ADDISON disease, myasthenia gravis and/or juvenile diabetes mellitus. Preferred intermediary autoimmune diseases are GOODPASTURE syndrome, autoimmune hemolytic anemia, autoimmune leukopenia, idiopathic thrombocytopenia, pemphigus vulgaris, sympathetic ophthalmia, primary bile cirrhosis, autoimmune hepatitis, colitis ulcerosa and/or SJÖGREN syndrome. Preferred systemic autoimmune diseases are rheumatoid arthritis, rheumatic fever, systemic lupus erythematodes, dermatomyositis/polymyositis, progressive systemic sclerosis, WEGENER granulomatosis, panarteritis nodosa and/or hypersensitivity angiitis. Typical autoimmune diseases are thyrotoxicosis, thyroid-caused myxedema, HASHIMOTO thyroiditis, generalized endocrinopathy, pernicious anemia, chronic gastritis type A, diseases of single or all corpuscular elements of the blood (for example, autoimmune hemolytic anemia, idiopathic thrombocytopenia or thrombocytopathy; idiopathic leukopenia or agranulocytosis), pemphigus vulgaris and pemphigoid, sympathetic ophthalmia, and numerous forms of uveitis, primarily biliary liver cirrhosis and chronic aggressive autoimmune hepatitis, diabetes mellitus type I, CROHN disease and colitis ulcerosa, SJÖGREN syndrome, ADDISON disease, lupus erythematodes disseminatus and discoid form of said disease, as dermatomyositis and scleroderma, rheumatoid arthritis (=primarily chronic polyarthritis), antiglomerular basement membrane nephritis. The basis is an aggressive immune reaction due to breakdown of the immune tolerance to self-determinants and a reduction of the activity of T suppressor cells (with lymphocyte marker T8) or an excess of T helper cells (with lymphocyte marker T4) over the suppressor cells; furthermore, formation of autoantigens is possible e.g. by coupling of host proteins to haptens (e.g. drugs), by ontogenetic tissue not developing until self-tolerance has developed, by protein components demasked as a result of conformational changes of proteins in connection with e.g. infection by viruses or bacteria; and by new proteins formed in connection with neoplasias.

Septicemic diseases in the meaning of the invention are diseases due to continuous or periodic invasion of pathogenic bacteria and/or their toxins from a focus of disease and their spreading on the lymph-blood route to form a general or local infection.

Septicemia in the meaning of the invention is preferably wound septicemia (phlegmon, thrombophlebitis, lymphangitis), puerperal septicemia (in case of puerperal fever), otogenic septicemia (in case of otitis media), tonsil-logenic septicemia (in case of angina, peritonsillitis), cholangitic septicemia (in case of purulent cholecystitis, cholangitis), pylephlebitic septicemia (in case of pylephlebitis) umbilical septicemia (in case of omphalitis etc.), urosepticemia, as well as dental granuloma. Septicemia in the meaning of the invention can be acute to highly acute (foudroyant), subacute (e.g. as endocarditis lenta) or chronic, and of course, can also be neonatal septicemia.

Therefore, septicemias in the meaning of the invention are all pathogenic changes in a patient which can be associated with intermittent fever and cold chills, with spleen tumor, toxic reactions or damage of the bone marrow or blood (polynuclear leukocytosis, anemia, hemolysis, thrombocytopenia) or with pathogenic reactions in the heart and vasomotor nerve (tachycardia, centralization of the blood circulation, edemas, oliguria; possibly shock) or in the digestive tract (dry, coated tongue, diarrhea), or with septicopyemia (pyemia with formation of septic infarction and metastatic abscess).

In the meaning of the invention, preferred diseases associated with a deficiency of the cellular immune system also include: AIDS, acne, albuminuria (proteinuria), alcohol withdrawal syndrome, allergies, alopecia (loss of hair), ALS (amyotrophic lateral sclerosis), Alzheimer's disease, retinal macula senile degeneration, anemia, anxiety syndrome, anthrax (milzbrand) aortic sclerosis, occlusive arterial disease, arteriosclerosis, arterial occlusion, arteriitis temporalis, arteriovenous fistula, asthma, respiratory insufficiency, autoimmune disease, prolapsed intervertebral disc, inflammation of the peritoneum, pancreatic cancer, Becker muscular dystrophy, benign prostate hyperplasia (BPH), bladder carcinoma, hemophilia, bronchial carcinoma, breast cancer, BSE, chlamydia infection, chronic pain, cirrhosis, commotio cerebri (brain concussion), Creutzfeld-Jacob disease, intestinal carcinoma, intestinal tuberculosis, depression, diabetes insipidus, diabetes mellitus, diabetes mellitus juvenilis, diabetic retinopathy, Duchenne muscular dystrophia, duodenal carcinoma, dystrophia musculorum progressiva, dystrophia, Ebola, eczema, erectile dysfunction, obesity, fibrosis, cervix cancer, uterine cancer, cerebral hemorrhage, encephalitis, loss of hair, hemiplegia, hemolytic anemia, hemophilia, urinary incontinence, pet allergy (animal hair allergy), skin cancer, herpes zoster, cardiac infarction, cardiac insufficiency, cardiovalvulitis, cerebral metastases, cerebral stroke, cerebral tumor, testicle cancer, ischemia, Kahler's disease (plasmocytoma), polio (poliomyelitis), rarefaction of bone, colon carcinoma, contact eczema, palsy, liver cirrhosis, leukemia, pulmonary fibrosis, lung cancer, pulmonary edema, lymph node cancer, (Morbus Hodgkin), lymphogranulomatosis, lymphoma, lyssa, gastric carcinoma, meningitis, mucoviscidosis (cystic fibrosis), multiple sclerosis (MS), myocardial infarction, neurodermitis, neurofibromatosis, neuronal tumors, kidney cancer (kidney cell carcinoma), osteoporosis, pancreas carcinoma, pneumonia, polyarthritis, polyneuropathies, potency disorders, progressive systemic sclerosis (PSS), prostate cancer, rectum carcinoma, pleurisy, craniocerebral trauma, vaginal carcinoma, sinusitis, esophagus cancer, tremor, tuberculosis, tumor pain, burns/scalds, intoxications, viral meningitis, menopause, soft-tissue sarcoma, soft-tissue tumor, cerebral blood circulation disorders, CNS tumors.

In a preferred embodiment the cancerous disease or tumor being treated or prevented is selected from the group of cancerous diseases or tumor diseases of the ear-nose-throat region, of the lungs, mediastinum, gastrointestinal tract, urogenital system, gynecological system, breast, endocrine system, skin, bone and soft-tissue sarcomas, mesotheliomas, melanomas, neoplasms of the central nervous system, cancerous diseases or tumor diseases during infancy, lymphomas, leukemias, paraneoplastic syndromes, metastases with unknown primary tumor (CUP syndrome), peritoneal carcinomatoses, immunosuppression-related malignancies and/or tumor metastases.

More specifically, the tumors may comprise the following types of cancer: adenocarcinoma of breast, prostate and colon; all forms of lung cancer starting in the bronchial tube; bone marrow cancer, melanoma, hepatoma, neuroblastoma; papilloma; apudoma, choristoma, branchioma; malignant carcinoid syndrome; carcinoid heart disease, carcinoma (for example, Walker carcinoma, basal cell carcinoma, squamobasal carcinoma, Brown-Pearce carcinoma, ductal carcinoma, Ehrlich tumor, in situ carcinoma, cancer-2 carcinoma, Merkel cell carcinoma, mucous cancer, non-parvicellular bronchial carcinoma, oat-cell carcinoma, papillary carcinoma, scirrhus carcinoma, bronchio-alveolar carcinoma, bronchial carcinoma, squamous cell carcinoma and transitional cell carcinoma); histiocytic functional disorder; leukemia (e.g. in connection with B cell leukemia, mixed-cell leukemia, null cell leukemia, T cell leukemia, chronic T cell leukemia, HTLV-II-associated leukemia, acute lymphocytic leukemia, chronic lymphocytic leukemia, mast cell leukemia, and myeloid leukemia); malignant histiocytosis, Hodgkin disease, non-Hodgkin lymphoma, solitary plasma cell tumor; reticuloendotheliosis, chondroblastoma; chondroma, chondrosarcoma; fibroma; fibrosarcoma; giant cell tumors; histiocytoma; lipoma; liposarcoma; leukosarcoma; mesothelioma; myxoma; myxosarcoma; osteoma; osteosarcoma; Ewing sarcoma; synovioma; adenofibroma; adenolymphoma; carcinosarcoma, chordoma, craniopharyngioma, dysgerminoma, hamartoma; mesenchymoma; mesonephroma, myosarcoma, ameloblastoma, cementoma; odontoma; teratoma; thymoma, chorioblastoma; adenocarcinoma, adenoma; cholangioma; cholesteatoma; cylindroma; cystadenocarcinoma, cystadenoma; granulosa cell tumor; gynadroblastoma; hidradenoma; islet-cell tumor; Leydig cell tumor; papilloma; Sertoli cell tumor, theca cell tumor, leiomyoma; leiomyosarcoma; myoblastoma; myoma; myosarcoma; rhabdomyoma; rhabdomyosarcoma; ependymoma; ganglioneuroma, glioma; medulloblastoma, meningioma; neurilemmoma; neuroblastoma; neuroepithelioma, neurofibroma, neuroma, paraganglioma, non-chromaffin paraganglioma, angiokeratoma, angiolymphoid hyperplasia with eosinophilia; sclerotizing angioma; angiomatosis; glomangioma; hemangioendothelioma; hemangioma; hemangiopericytoma, hemangiosarcoma; lymphangioma, lymphangiomyoma, lymphangiosarcoma; pinealoma; cystosarcoma phylloides; hemangiosarcoma; lymphangiosarcoma; myxosarcoma, ovarial carcinoma; sarcoma (for example, Ewing sarcoma, experimentally, Kaposi sarcoma and mast cell sarcoma); neoplasms (for example, bone neoplasms, breast neoplasms, neoplasms of the digestive system, colorectal neoplasms, liver neoplasms, pancreas neoplasms, hypophysis neoplasms, testicle neoplasms, orbital neoplasms, neoplasms of the head and neck, of the central nervous system, neoplasms of the hearing organ, pelvis, respiratory tract and urogenital tract); neurofibromatosis and cervical squamous cell dysplasia.

In another preferred embodiment the cancerous disease or tumor being treated or prevented is selected from the group of tumors of the ear-nose-throat region, comprising tumors of the inner nose, nasal sinus, nasopharynx, lips, oral cavity, oropharynx, larynx, hypopharynx, ear, salivary glands, and paragangliomas, tumors of the lungs comprising non-parvicellular bronchial carcinomas, parvicellular bronchial carcinomas, tumors of the mediastinum, tumors of the gastrointestinal tract, comprising tumors of the esophagus, stomach, pancreas, liver, gallbladder and biliary tract, small intestine, colon and rectal carcinomas and anal carcinomas, urogenital tumors comprising tumors of the kidneys, ureter, bladder, prostate gland, urethra, penis and testicles, gynecological tumors comprising tumors of the cervix, vagina, vulva, uterine cancer, malignant trophoblast disease, ovarial carcinoma, tumors of the uterine tube (Tuba Faloppii), tumors of the abdominal cavity, mammary carcinomas, tumors of the endocrine organs, comprising tumors of the thyroid, parathyroid, adrenal cortex, endocrine pancreas tumors, carcinoid tumors and carcinoid syndrome, multiple endocrine neoplasias, bone and soft-tissue sarcomas, mesotheliomas, skin tumors, melanomas comprising cutaneous and intraocular melanomas, tumors of the central nervous system, tumors during infancy, comprising retinoblastoma, Wilms tumor, neurofibromatosis, neuroblastoma, Ewing sarcoma tumor family, rhabdomyosarcoma, lymphomas comprising non-Hodgkin lymphomas, cutaneous T cell lymphomas, primary lymphomas of the central nervous system, morbus Hodgkin, leukemias comprising acute leukemias, chronic myeloid and lymphatic leukemias, plasma cell neoplasms, myelodysplasia syndromes, paraneoplastic syndromes, metastases with unknown primary tumor (CUP syndrome), peritoneal carcinomatosis, immunosuppression-related malignancy comprising AIDS-related malignancy such as Kaposi sarcoma, AIDS-associated lymphomas, AIDS-associated lymphomas of the central nervous system, AIDS-associated morbus Hodgkin and AIDS-associated anogenital tumors, transplantation-related malignancy, metastasized tumors comprising brain metastases, lung metastases, liver metastases, bone metastases, pleural and pericardial metastases, and malignant ascites.

In another preferred embodiment the cancerous disease or tumor being treated or prevented is selected from the group comprising mammary carcinomas, gastrointestinal tumors, including colon carcinomas, stomach carcinomas, pancreas carcinomas, colon cancer, small intestine cancer, ovarial carcinomas, cervical carcinomas, lung cancer, prostate cancer, kidney cell carcinomas and/or liver metastases.

The invention also relates to the use of the composition of the invention in procedures for the prophylaxis and/or therapy of persons, animals and/or patients with pathogenic modifications and/or cellular immunodeficiencies, especially cancer, sepsis, allergic reactions associated with a cytostatic agent therapy, chemotherapy and/or radiotherapy and/or as prophylaxis and/or therapy in connection with accidents involving nuclear, biological, chemical and/or radioactive substances and/or materials.

The invention also relates to a kit and to the use thereof in medicine. In a preferred fashion, the compounds of the invention or the kit comprising same is used in a combination therapy, especially in the treatment of tumors. In a particularly preferred fashion, said combination therapy comprises a chemotherapy, a treatment with cytostatic agents and/or a radiotherapy. In a particularly preferred embodiment of the invention the combination therapy is a biologically specific form of therapy, and in a particularly preferred fashion, said form of therapy is an immune therapy. Furthermore, in a particularly preferred fashion the combination therapy comprises a gene therapy and/or a therapy using a compound according to the invention. Various combination therapies, especially for the treatment of tumors, are well-known to those skilled in the art. For example, a treatment with cytostatic agents or irradiation of a particular tumor area can be envisaged within the scope of a combination therapy, and this treatment is combined with a gene therapy, using the compounds of the invention as anticancer agents. Accordingly, the use of the compounds according to the invention for increasing the sensitivity of tumor cells to cytostatic agents and/or radiation can be particularly preferred. Furthermore, a preferred use of the compounds according to the invention is in inhibiting the vitality, the proliferation rate of cells and/or inducing apoptosis and cell cycle arrest.

Without intending to be limiting, the invention will be explained in more detail with reference to the following examples.

### EXAMPLES

General representation of the production of the composition of the invention using the method according to the invention

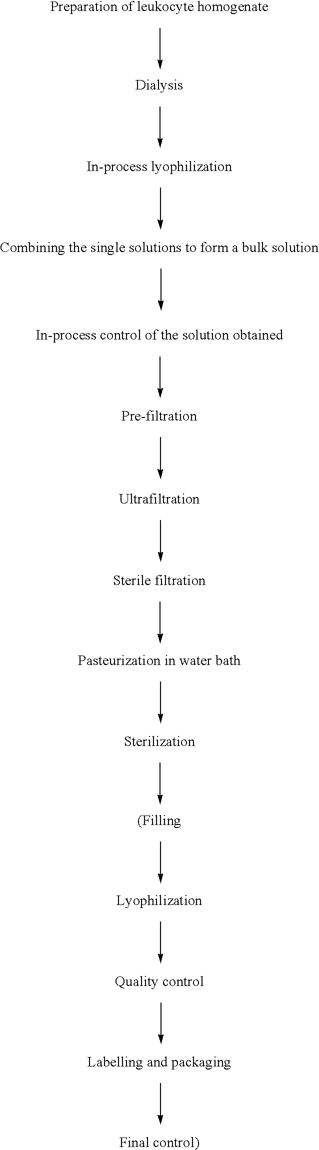

Description of the Method

1. Preparation of a Concentrate Comprising Cellular Blood Components, e.g. Leukocytes or Erythrocytes

First of all, a homogenate of selected cells is prepared, e.g. by means of repeated freeze-thaw cycles or ultrasonic treatment of the cells, or by a combination of both processes. Thereafter, the individual volumes are pooled.

2. Dialysis of the Homogenate

The dialysis is performed in the form of a column or membrane dialysis where all particles having a size of more than 10 kDa are removed.

3. Intermediate Lyophilization

The dialyzed product is concentrated by means of lyophilization, said lyophilization being carried out using standard procedures.

4. Preparation of a Provisional Solution of the Composition According to the Invention

The lyophilized product is filled up with 2 ml of aqua.

5. Intermediate Control

The intermediate control is performed using absorption measurement in a spectral range of from 260 to 280 nm.

6. Prefiltration

Filtration is effected through a Millipore RA membrane (1.2 μm) using an AP 15 prefilter.

7. Ultrafiltration:

Ultrafiltration is effected through a PTGC membrane in a Millipore cartridge system with an exclusion limit of 10 kDa.

8. Sterilization by Filtration

Sterilization by filtration is effected using a Millipore GS filter (0.22 μm).

9. Heat Inactivation

The solution of the invention is pasteurized in single vessels in a water bath at a temperature of 60° C. for 10 hours.

10. Aliquoting

The liquid composition is subjected to automatic aliquoting under sterile conditions (from 2 liters of total solution into 5 ml vials).

11. Lyophilization:

Lyophilization is effected using standard procedures. Filling of the single aliquots is done under a nitrogen atmosphere with cooling.

The composition of the invention was also tested in vivo in various animal systems. Using the rosette test on guinea pig T lymphocytes, the state of cellular immunity under the influence of the composition according to the invention in combination with Oxoplatin, Campto, Taxol and Eloxatin was investigated. In each test, the improvement of the T lymphocyte state in immunosuppressed guinea pigs after administration of the composition according to the invention was tested. Prior to testing, the level of T lymphocytes in the guinea pigs was determined using the rosette test, and subsequently, the decrease in the amount of T lymphocytes caused by the immunosuppressant Azathioprin was determined. A second determination of the T lymphocytes was made seven days after application of Azathioprin. This was followed by subcutaneous application of the composition of the invention into the laboratory animals. The last determination of the T lymphocyte count in the guinea pigs was made 14 to 19 days after application of the composition according to the invention.

It was found in these in vivo tests that the production of rosettes dropped by about 30% following application of Oxoplatin. The average increase of rosette formation after application of the composition according to the invention was 27% in a single administration of Oxoplatin and 23% in those cases where the animals had been treated beforehand with the double amount of Oxoplatin.

In tests using Campto and the composition it was found that the production of rosettes was reduced by 23% after application of Campto and increased by 26% after application of said composition.

When using Taxol and the composition, there was a drop in the production of rosettes by 16% and an increase by 23% after administration of the composition according to the invention.

The formation of rosettes following administration of Eloxatin dropped by 22% and increased by 29% after administration of the composition according to the invention.

Also, clinical studies on humans were conducted to further verify the effects of the composition according to the invention:

Five female patients suffering from sclerosis (PSS) were treated with the composition. Normalization of the rosette cell count was detected. In addition to normalized cellular immunity, an improvement of the acral circulation was detected. The clinical effect of the composition in normalizing the rosette cell level resulted in an increase from 38% to 64%.

Furthermore, the composition according to the invention was investigated on human patients suffering from psoriasis vulgaris and a form of arthritis derived from psoriasis. The patients were administered with three doses of the composition of the invention at weekly intervals, a single dose comprising 4 mg of composition in 2 ml. Six months after initiating the therapy, complete disappearance of the symptoms of the two above-mentioned diseases was found in three out of eight patients. In the other five cases a significant improvement of the clinical picture was observed. The immunologic improvement of the overall situation was associated with an improvement of the total relevant clinical data. The average level of rosette cells in the patients was 33% prior to starting therapy and increased to 67% at the end of the therapy.

In another clinical test the composition according to the invention was used in female patients diagnosed as suffering from systemic lupus erythematosus (SLE). An improvement of the clinical picture was determined in the patients. In addition, some patients had an infection with candidal pathogens. Similarly, an improvement of the overall clinical picture was determined in these patients so that initiation of a standard therapy was possible.

Furthermore, investigations were carried out with 35 patients diagnosed as suffering from anterior uveitis. The treatment period was 35 to 85 months, and the success of the therapy was re-investigated during a period of 145 to 195 months. It was possible to demonstrate that the above long-term application prevented recurrence of the anterior uveitis in 90% of the patients. In those patients where recurrence of the disease had been diagnosed, the disease appeared in a very mild form. About 5% of the patients failed to respond to the treatment.

Various cancer cell lines were tested with the composition according to the invention, in which tests single dilution steps were investigated, beginning with 500 μg/ml. The best effects were determined at a concentration of 30 μg/ml.

| Cell line | | % inhibition |
|---|---|---|
| Colo205 | Colon | 10.9 |
| BT20 | Breast | 0 |
| PC3 | Prostate | −7.4 |
| SK-MTC | Thyroid | 16.6 |
| J82 | Bladder | 8.7 |
| WI38 | Fibroblasts | 14.5 |
| A431 | Carcinoma | 13.3 |
| HT29 | Colon | 9.4 |
| PANC1 | Pancreas | 17.0 |
| MIAPaCa2 | Pancreas | 34.9 |
| LNCaP | Prostate | 30.8 |

The composition according to the invention has an antiproliferative effect on most of these cell lines, especially in MIAPaCa2 pancreas cancer cells and LNCaP hormone-sensitive prostate cancer cell lines.

1.-19. (canceled)

20. A method for producing a composition for treating cellular immunodeficiency in a patient comprising:
homogenizing cellular blood components to produce a homogenized product,
lyophilizing the homogenized product, and
removing components with a molecular weight of more than 10,000 Da.

21. The method according to claim 20, wherein said blood components are leukocytes.

22. The method according to claim 21, wherein a leukocyte concentrate is initially produced, which is subsequently dialyzed, followed by pre-filtration, ultrafiltration and, optionally, subsequent pasteurization.

23. The method according to claim 20, wherein
a) said cellular blood components are homogenized using a freeze-thaw cycle and/or ultrasonic treatment;
b) a homogenate obtained according to a) is lyophilized;
c) a lyophilizate obtained according to b) is pre-filtered;
d) a pre-filtrate obtained according to c) is ultrafiltrated;
e) an ultrafiltrate obtained according to d) is sterile filtrated;
f) a sterile filtrate obtained according to e) is pateurized;
g) a pasteurized product according to f) is sterilized; and
h) a sterile product according to g) is lyophilized.

24. (cancelled)

25. The method according to claims 20, wherein the method further comprises formulating the composition obtained or a derivative or a homologue thereof with a pharmaceutically acceptable carrier.

26. A composition for treating cellular immunodeficiency in a patient comprising a lyophilized homogenate of cellular blood components having a molecular weight of less than or equal to 10,000 Da.

27. The composition of claim 26, wherein said cellular blood components are leukocytes.

28. The composition according to claim 27 comprising ubiquitin-specific protease 32, positive cofactor 2 glutamine/Q-rich associated protein, cadherin, transcription factor GATA-2, putative chromatin structure regulator, CUE domain containing 1, SUPT6H protein, interleukin-18 receptor 1 precursor, interleukin-1 receptor-like protein and mucin 4 and/or tenascin M, transient receptor potential cation channel, ectonucleotide pyrophosphatase/phosphodiesterase, SWI/SNF-related matrix-associated actin-dependent regulator of chromatin, SWI/SNF chromatin-modulating complex subunit OSA1B120, OSA1 nucleoprotein, ATPase, H⁺/K⁺ change polypeptide, zinc-finger protein 174, Ig heavy chain V region, ADAMTS-3 precursor/desintegrin-like and metalloprotease, coagulation factor II (thrombin) receptor-like 3, diacylglycerol kinase-theta, p250R, SWI/SNF chromatin-remodulating complex subunit OSA2, metallo-phospoesterase, AMP deaminase, α-mannosidase, cadherin-9, Nik-related kinase, a-1 B-adrenergic receptor, BRCA1-associated protein, CD99 antigen-like 2 isoform E3-E4, SWHCF-comprising peptide and/or FAT-like cadherin-FATJ, ATP binding cassette, nucleoporin 153 kDa, ELK3 protein, protein ELK-3 ETS domain, ATPase, copper-transporting protein kinase, protein-tyrosine phosphatase, wingless type MMTV integration site family, MYC binding protein 2, cullin 7, dissolved carrier family 5 (sodium iodide symporter) member 5, glutamate-rich WD repeat containing 1, MAP kinase-interacting serine/threonine kinase 1, ATP binding cassette, NOV plexin A1 protein and/or E3 ubiquitin ligase SMURF2, acyl-CoA synthetase, estrogen sulfotransferase, 2,4-dienoyl CoA reductase 1 precursor, 4-enoyl CoA reductase, claudin 10 isoform b, DMBT1, extracellular linker domain containing 1, lymphatic vessel endothelial cell-specific hyaluron receptor LYVE-1, Rho-GTPase-activating protein 8 isoform 2, desintegrin-like and metalloprotease (reprolysine type) with thrombospondin type 1 motif, Ig heavy chain V region, AS12 protein, mitochondrial ribosomal protein S9, 28S ribosomal protein S9, protein kinase substrate MK2S4, NP220, putative G protein-coupled receptor, dynein, axonemal heavy polypeptide 5, N-acylphosphatidyl ethanolamine-hydrolyzing phospholipase D, leukotriene B4 receptor, G protein-coupled receptor 16, proprotein convertase subtilisin/kexin type 1 inhibitor CMKRL1, dual-specific tyrosine phosphorylation-regulated kinase 3, regulatory erythroid kinase (long form), DYRK3 protein, Ig lambda chain V-VII region (Mot)—human, glutathione reductase, mitochondrial precursor, collagen alpha 1 (XVI) chain precursors, 11-β-hydroxysteroid dehydrogenase 1, insulin receptor substrate 2, Vault poly(ADP-ribose) polymerase (VPARP), calcium/calmodulin-dependent 3′,5′-cyclic nucleotides, zinc-finger protein 161, H2.0-like homeobox-1, H2.0 (drosophila)-like homeobox-1 and/or dedicator of cytokinesis 6.

29. The composition according to claim 28, comprising interleukin-18 receptor 1 precursor, interleukin-1 receptor-like protein and mucin 4, transient receptor potential cation channel, ectonucleotide pyrophosphatase/phosphodiesterase, SWI/SNF-related matrix-associated actin-dependent regulator of chromatin, SWI/SNF chromatin-modulating complex subunit OSA1 B120, OSA1 nucleoprotein, MYC binding protein 2, cullin 7, dissolved carrier family 5 (sodium iodide symporter) member 5, glutamate-rich WD repeat containing 1, MAP kinase-interacting serine/threonine kinase 1, ATP binding cassette, DMBT1, extracellular linker domain containing 1, lymphatic vessel endothelial cell-specific hyaluron receptor LYVE-1, Rho-GTPase-activating protein 8 isoform 2, desintegrin-like and metalloprotease (reprolysine type) with thrombospondin type 1 motif, AS12 protein, mitochondrial ribosomal protein S9, 28S ribosomal protein S9, protein kinase substrate MK2S4, NP220, putative G protein-coupled receptor, dynein, axonemal, heavy polypeptide 5, N-acylphosphatidyl ethanolamine-hydrolyzing phospholipase D, leukotriene B4 receptor, G protein-coupled receptor 16, proprotein convertase subtilisin/kexin type 1 inhibitor CMKRL1, dual-specific tyrosine phosphorylation-regulated kinase 3, regulatory erythroid kinase (long form), DYRK3 protein, Ig lambda chain V-VII region (Mot)—human.

30. A pharmaceutical agent comprising the composition according to claims 27, optionally together with a pharmaceutically acceptable carrier.

31. The pharmaceutical agent according to claim 30, wherein the carrier is selected from the group consisting of fillers, disintegrants, binders, humectants, extenders, dissolution retarders, absorption enhancers, wetting agents, adsorbents, lubricants and combinations thereof.

32. The pharmaceutical agent according to claim 30, wherein said agent is a capsule, a tablet, a coated tablet, a suppository, an ointment, a cream, an injection solution and/or an infusion solution.

33. The pharmaceutical agent according to claim 30, wherein said agent is a vaginal and/or rectal suppository, pad and/or foam.

34. The pharmaceutical agent according to claim 30, characterized in that said agent is enclosed in liposomes, siosomes and/or niosomes.

35. A kit comprising a composition according to claim 27, for use as a drug, optionally together with information relating to the combination and/or handling of the components of the kit.

36. Method for treating pathogenic modifications of the cellular immunity in persons, animals and/or a patient comprising administering the pharmaceutical agent according to claim 30 in a pathogenic modifications of the cellular immunity treating effective amount.

37. The method of claim 36, wherein said pathogenic modification of the cellular immunity is a cellular immunodeficiency.

38. The method of claim 37, wherein the cellular immunodeficiency is a cellular immunodeficiency according to ICD10 code D84.8.

39. The method of claim 36, wherein the pharmaceutical composition is contacted with the patient in connection with a cytostatic agent therapy, chemotherapy and/or radiotherapy.

40. The method of claim 36, wherein said pharmaceutical agent is administered orally, vaginally, rectally, nasally, topically, subcutaneously, intravenously, intramuscularly, intraperitoneally via injections and/or over infusions.

41. The method of claim 36, wherein the pharmaceutical composition is contacted with persons, animals and/or a patient before and/or after serious accidents.

42. The method of claim 41, wherein said persons, animals and/or patient is/are is in need of prophylaxis of secondary deficiencies.

43. The method of claim 42, wherein said secondary deficiency is septicemia.

44. The method of claim 36, wherein said persons, animals and/or patient is/are in need of prophylaxis and therapy in connection with accidents with nuclear, biological, chemical and/or radioactive substances and/or materials.

45. The method of claim 44, wherein said persons and/or animals have come in contact with nuclear, biological, chemical and/or radioactive substances and/or materials.

46. The method according to claims 20, wherein lyophilization is effected with addition of solutions.

47. The method of claim 46, wherein said solutions are buffers, salts, vitamins, sugar derivatives, enzymes and/or vegetable extracts.
